(19) United States
(12) Plant Patent Application Publication
(10) Pub. No.: US 2021/0059089 P1
(43) Pub. Date: Feb. 25, 2021

(54) Apple Tree Named 'Bingo Gala'

(50) ;

(71) Applicant: Golden Joy Bingo (Pty.) Ltd., Grabouw (ZA)

(72) Inventor: Jan Dawid Wiegman, Grabouw (ZA)

(21) Appl. No.: 16/873,936

(22) Filed: Aug. 17, 2020

Related U.S. Application Data
(60) Provisional application No. 62/922,687, filed on Aug. 21, 2019.

(51) Int. Cl. A01H 6/74

(52) U.S. Cl. PLT/161

(57) ABSTRACT

‘Bingo Gala’ is a new apple variety discovered as a limb mutation of ‘Tenroy Gala.’ The fruit of ‘Bingo Gala’ is notable for its cherry-red overcolor, which consistently covers about eighty percent of the fruit surface. ‘Bingo Gala’ can be harvested in one pick.

Latin name: Malus domestica.

Variety denomination: ‘Bingo Gala’.

### BACKGROUND OF THE VARIETY

‘Bingo Gala’ is a new and distinct variety of ‘Gala’-type apple tree. The new variety is a spontaneous limb mutation of ‘Tenroy Gala’ (U.S. Plant Pat. No. 4,121, expired). ‘Bingo Gala’ was first observed in 2012 at Caledon, Republic of South Africa, in a commercial orchard planted with ‘Tenroy Gala’ trees, and was first asexually reproduced by grafting in 2012 in the same location. The variety has since been asexually reproduced by budding, and has been observed to remain true to type over successive asexually propagated generations.

### BRIEF DESCRIPTION OF THE VARIETY

‘Bingo Gala’ was first selected for its attractive fruit, which has a cherry-red blush over about 80% of the fruit surface. This desirable coloration has been found to occur very consistently, allowing harvest to be accomplished in a single pick. ‘Bingo Gala’ is distinguished from ‘Tenroy Gala’ and ‘Bigbucks’ (U.S. Plant Pat. 29,365) as set forth below.

TABLE 1
Comparison of ‘Bingo Gala’ to Parent ‘Tenroy Gala’

| Characteristic | ‘Bingo Gala’ | ‘Tenroy Gala’ |
|---|---|---|
| Fruit overcolor | Red to bright red | Red to purple |
| Amount of overcolor | Blush over about 80% | Up to about 50% |
| | of surface area | of surface area |
| Fruit stem color | Red | Purple |
| Leaf color near base | Red | Purple |
| Nursery tree size | 20 cm shorter than | 20 cm taller than |
| | ‘Tenroy Gala’ | ‘Bingo Gala’ |

TABLE 2
Comparison of ‘Bingo Gala’ to ‘Bigbucks’

| Characteristic | ‘Bingo Gala’ | ‘Bigbucks’ |
|---|---|---|
| Fruit overcolor | Red to bright red | Dark red |
| Amount of overcolor | Blush over about 80% | Solid over about 90% to |
| | of surface area | 100% of surface area |
| Fruit stem color | Red | Purple |
| Leaf color near base | Red | Purple |

### BRIEF DESCRIPTION OF THE PHOTOGRAPHS

The attached photographs were taken during the 2018 and 2019 growing seasons near Caledon, Republic of South Africa. The trees shown were planted in 2013 on ‘M793’ rootstock (not patented).

FIG. 1 shows ‘Bingo Gala’ trees;

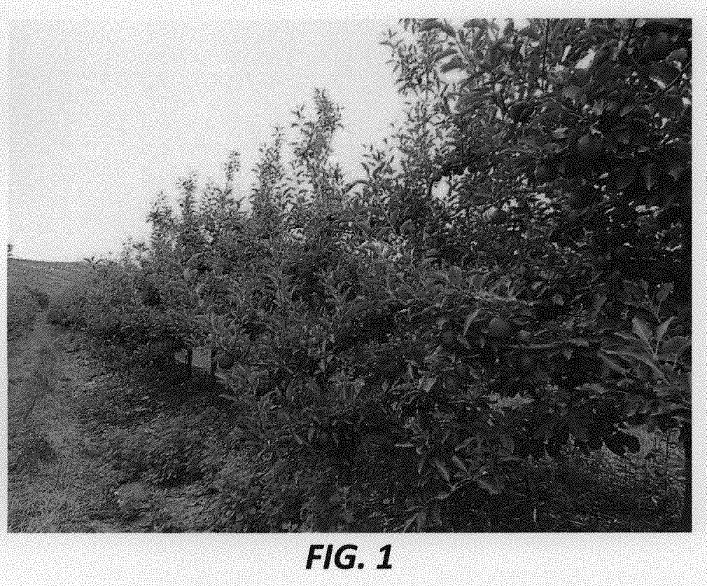

FIG. 2 shows ‘Bingo Gala’ fruit and leaves on the tree;

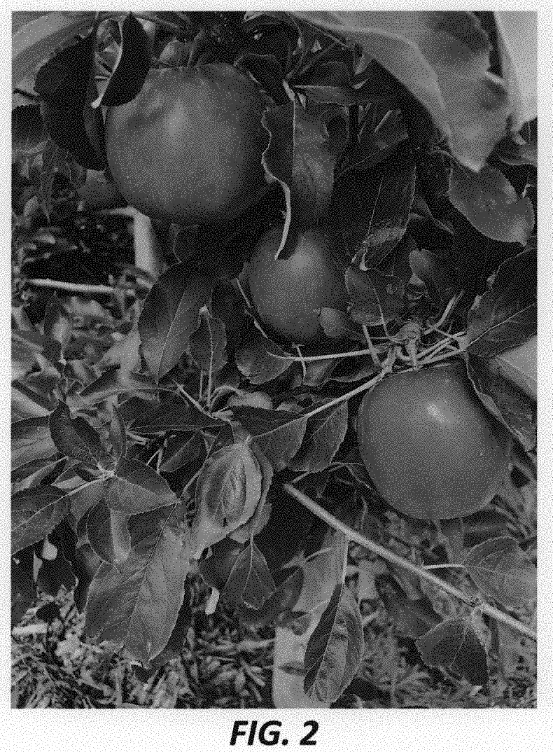

FIG. 3 shows ‘Bingo Gala’ fruit and leaves on the tree;

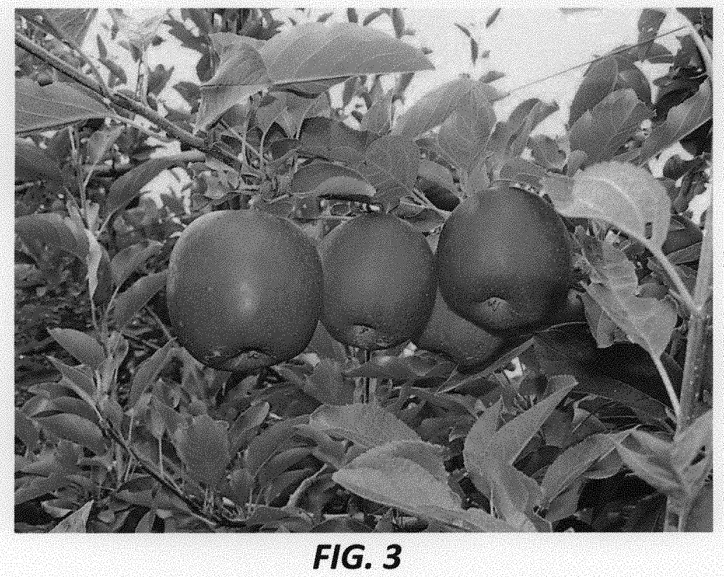

FIG. 4 shows ‘Bingo Gala’ fruit and leaves on the tree;

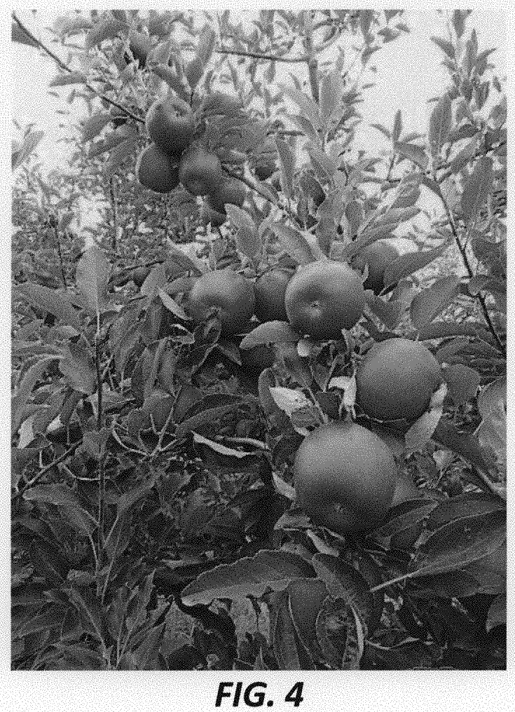

FIG. 5 shows fruit of ‘Bigbucks’ (left) and ‘Bingo Gala’ (right);

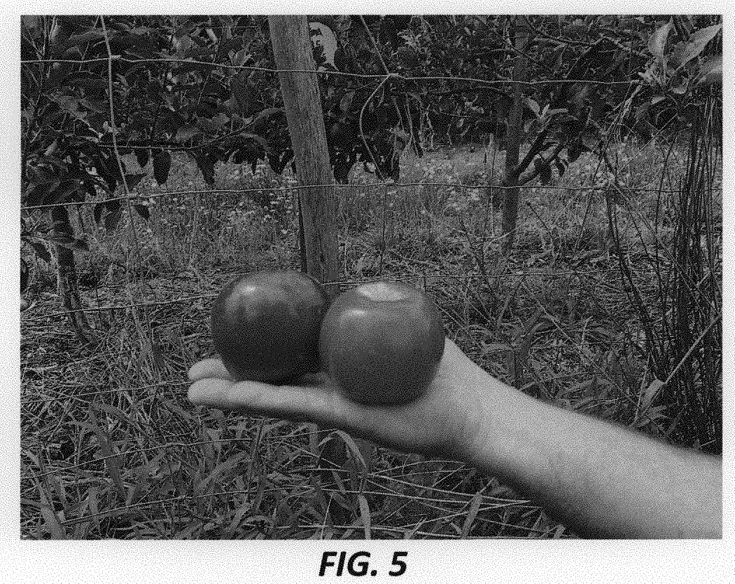

FIG. 6 shows fruit of ‘Bingo Gala’ (left) and ‘Bigbucks’ (right);

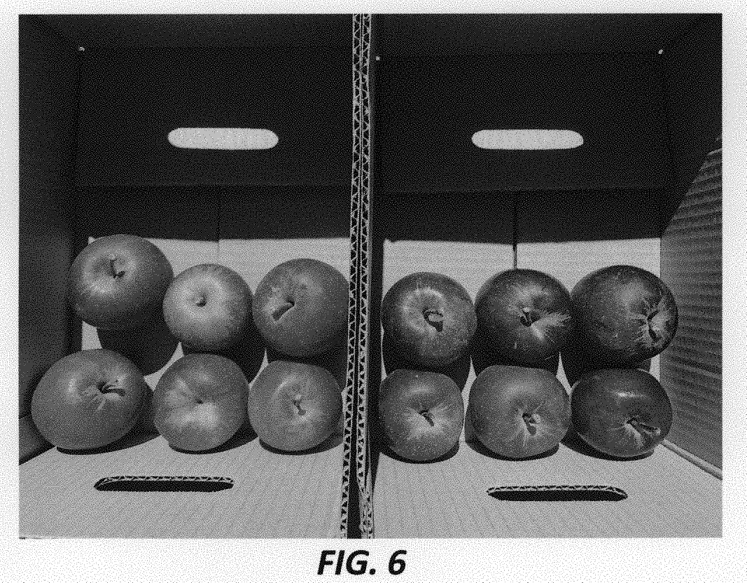

FIG. 7 shows a whole ‘Bingo Gala’ fruit; and

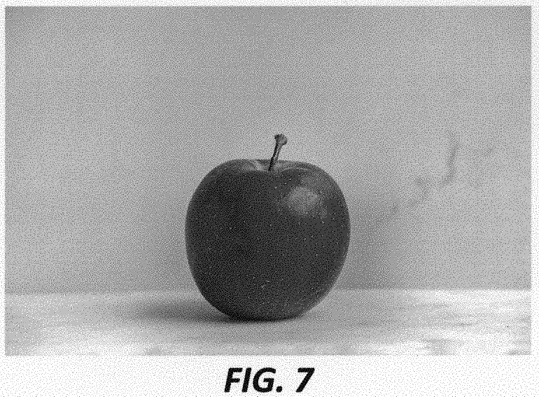

FIG. 8 shows whole and sectioned ‘Bingo Gala’ fruit and leaves.

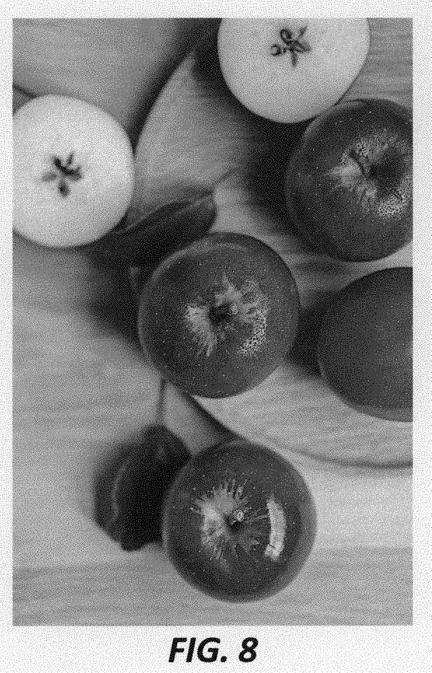

### DETAILED BOTANICAL DESCRIPTION OF THE VARIETY

The following botanical description is based primarily on observations made during the 2018 and 2019 growing seasons near Caledon, Republic of South Africa. The described trees were planted in 2013 on ‘M793’ rootstock. It should be understood that the characteristics described will vary somewhat depending upon cultural practices and climatic conditions, and will vary with location and season.
Tree:

### Vigor.—Strong.

Type.—Ramified.
Habit.—Semi-upright.
Height.—3 m.
Trunk diameter at 30 cm above graft.—15 cm.
Bearing.—On shoots and spurs.
Bark texture.—Smooth.
Lenticels.—Not present on trunk.
Branch (fruiting branches located at around 1 m above the graft union):
   Length.—100 cm.
   Diameter.—12 mm.
   Crotch angle.—30° to 45°.
   Bark color.—Medium brown.
One year old shoot:
   Length.—50 cm.
   Diameter.—8mm.
   Pubescence.—Weak.
   Shoot color.—Medium brown.
   Internode length.—Short.
   Lenticel shape.—Elongated.
   Lenticel size.—4 mm×2 mm.
   Lenticel color.—Greyed white 156C.
Flowers:
   Bud shape.—Round conical.
   Quantity per spur.—5 to 6.
   Time of beginning of flowering (when tree presents 10% fully opened flowers).—Mid-season (Sept 20, 2019).
   Date of full bloom.—Mid-season (Oct. 15, 2019).
   Pollination requirement.—Pollinator required (‘Granny Smith’, ‘Golden Delicious’).
   Diameter of fully open flower.—Medium, 40 mm.
   Relative position of petal margin.—Not touching.
   Petal width.—19 mm.
   Petal color.—Medium pink.
Leaves:
   Shape.—Ovate to elliptic.
   Length.—Medium, 93 mm.
   Width.—Narrow, 51 mm.
   Length/width ratio.—Large, 1.8.
   Blade margin.—Serrate.
   Apex shape.—Acuminate.
   Base shape.—Cuneate.
   Color of upper surface.—Medium green.
   Attitude in relation to shoot.—Upwards.
Petiole:
   Length.—Long, 37 mm.
   Anthocyanin coloration.—Present, medium intensity.
Fruit:
   Quantity per cluster.—1 to 3.
   Diameter.—Medium, 72 mm.
   Height.—Medium, 67 mm.
   Weight.—182 g.
   Ratio of height to width.—Medium, 0.92.
   General shape in profile.—Globose.
   Position of maximum diameter.—Middle.
   Ribbing.—Absent or very weak.
   Crowning at calyx end.—Weak.
   Size of eye.—Medium.
   Aperture of eye.—Closed.
   Length of sepal.—Medium.
   Bloom of skin.—Medium.
   Greasiness of skin.—Absent.
   Background color of skin.—Yellow.
   Over color of skin.—Red.
   Amount of over color.—Very high.
   Intensity of over color.—High.
   Pattern of over color.—Only solid blush.
   Amount of russet around stalk cavity.—Absent or small.
   Amount of russet on cheeks.—Absent or small.
   Area of russet around eye basin.—Absent or small.
   Length of stalk.—Medium.
   Thickness of stalk.—Medium.
   Stalk color.—Red.
   Depth of stalk cavity.—Deep, 16 mm.
   Width of stalk cavity.—Narrow, 30 mm.
   Depth of eye basin.—Shallow, 7 mm.
   Width of eye basin.—Very narrow, 26 mm.
   Firmness of flesh.—Medium, 7.5 kg.
   Flesh texture.—Crisp.
   Aperture of locules.—Moderately open.
   Locule width.—18 mm.
   Aroma.—Good.
   Juiciness.—High.
   Brix.—15° Brix.
   Flesh color.—Cream.
   Quantity of seeds per fruit.—About 10, similar to ‘Tenroy Gala’.
   Eating quality of fruit.—Excellent.
   Maturity.—Early (early to mid-February; Feb. 21, 2019).
   Resistance to pests and diseases.—Typical of ‘Gala’-types.
   Market use.—Fresh.

I claim :

1. a new and distinct apple tree substantially as described and illustrated herein.
